(19) United States
(12) Patent Application Publication
(10) Pub. No.: US 2009/0242797 A1
(43) Pub. Date: Oct. 1, 2009

(54) SYSTEM AND METHOD FOR MULTI-MODE OPTICAL IMAGING

(75) Inventors: Siavash Yazdanfar, Niskayuna, NY (US); Thomas W. Karpen, Skaneateles, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company, Schenectady, NY (US)

(21) Appl. No.: 12/059,749

(22) Filed: Mar. 31, 2008

(51) Int. Cl. G01N 21/64; A61B 1/07

(52) U.S. Cl. 250/458.1; 600/178; 250/459.1

(57) ABSTRACT

A technique is provided for multi-mode optical imaging. The technique includes directing a visible light and an excitation light multiplexed in time towards a specimen. The excitation light is configured to induce luminescence in the specimen. The technique also includes detecting visible light scattered or reflected from the specimen and luminescent light emitted via luminescence via a single detector that is in synchronization with the one or more illumination sources.

### BACKGROUND

The invention relates generally to the field of imaging and more specifically, to the field of multi-mode optical imaging.

Various imaging techniques have been developed for use in a wide range of applications. For example, in modern healthcare facilities, imaging systems are often used for identifying, diagnosing, and treating physical conditions. Medical imaging systems may employ a variety of different techniques to image and visualize the internal structures and/or functional behavior (such as chemical or metabolic activity) of organs and tissues within a patient. Currently, a number of modalities exist for medical diagnostic and imaging systems, each typically operating on different physical principles to generate different types of images and information. These modalities include ultrasound systems, computed tomography (CT) systems, X-ray systems (including both conventional and digital or digitized imaging systems), positron emission tomography (PET) systems, single photon emission computed tomography (SPECT) systems, and magnetic resonance (MR) imaging systems.

Another imaging modality is optical imaging, which operates by propagating light of certain wavelengths at target and directly visualizing or generating an image based on the detected light. Based on the particular optical modality used, different wavelengths of light may be used to measure optical properties of tissue or generate an enhanced image of a region of interest for the physician.

An endoscope is an optical imaging device that provides real-time, high-resolution views of the interior of hollow organs and cavities. Although most endoscopes are designed for direct visual inspection with brightfield (white light) imaging, there has been a recent emergence of other detection modalities, including narrow band illumination, luminescence (e.g., fluorescence and phosphorescence), and imaging of light outside the visible wavelength range. For example, fluorescence endoscopy utilizes differences in the fluorescence response of normal tissue and abnormal tissue, such as in the detection and localization of such cancer. The fluorophores that are excited during fluorescence endoscopy may be exogenously applied agents that accumulate preferentially in disease associated tissues, or they may be the endogenous fluorophores that are present in all tissue. In the latter case, the fluorescence from the tissue is typically referred to as autofluorescence. Tissue autofluorescence is typically due to fluorophores with absorption bands in the UV and blue portion of the visible spectrum and certain emission bands in the green to red portions of the visible spectrum. In tissue states associated with early cancer, the green portion of the autofluorescence spectrum is appreciably suppressed. And, this spectral difference between disease and healthy tissue may be used to distinguish normal from suspicious tissue.

Endoscopes have been developed that can function in multiple modes such as in response to different wavelengths of light, such as white, narrow band, and luminescent. Typically, one or more light sources and detectors dedicated to each modality are placed at the user-interface end of a multi-mode endoscope. The light corresponding to each modality travels from the sources through fiber bundles to the tissue being imaged. The reflected and/or emitted light then travel through the fiber bundles from the tissue to the corresponding detectors.

However, the coherent fiber bundle has a limited coupling efficiency and transmission window, resulting in limited resolution, dead pixels, and other artifacts caused by the transmission through the fiber bundle. Distal chip approaches (detector being placed near the tissue being imaged) enable superior image quality, but are much more challenging due to limitations on the size of the device, constrained by tissue and cavity sizes, as well as patient comfort. Thus, adding a second imaging modality, such as fluorescence or infrared imaging, requires a larger probe as it requires an additional dedicated detector, increasing patient discomfort and damage to the probed tissue. Also, the use of two cameras in a multi-mode endoscope can lead to registration problems, thereby requiring periodic calibrations and adjustments.

It is therefore desirable to provide multi-mode endoscope systems that solve the problems of the prior art.

### BRIEF DESCRIPTION

Briefly, in accordance with one aspect of the technique, a system is provided for multi-mode optical imaging. The system includes one or more illumination sources for directing a visible light and an excitation light multiplexed in time towards a specimen. The excitation light is configured to induce luminescence in the specimen. The system also includes a single detector in synchronization with the one or more illumination sources for detecting visible light scattered or reflected from the specimen and luminescent light emitted via luminescence.

In accordance with another aspect of the technique, a multi-mode endoscope is provided. The multi-mode endoscope includes one or more illumination sources disposed within an endoscope body and configured to direct a visible light and an excitation light multiplexed in time towards a specimen via a fiber optic cable. The excitation light is configured to induce luminescence in the specimen. The multi-mode endoscope also includes a single detector disposed within an endoscope probe, in synchronization with the one or more illumination sources, and configured to detect visible light scattered or reflected from the specimen and luminescent light emitted via luminescence.

In accordance with a further aspect of the technique, a method is provided for multi-mode optical imaging. The method provides for directing a visible light and an excitation light multiplexed in time towards a specimen. The excitation light is configured to induce luminescence in the specimen. The method also provides for detecting visible light scattered or reflected from the specimen and luminescent light emitted via luminescence via a single detector that is in synchronization with the one or more illumination sources. Systems and computer programs that afford such functionality may be provided by the present technique.

### DRAWINGS

These and other features, aspects, and advantages of the present invention will become better understood when the following detailed description is read with reference to the accompanying drawings in which like characters represent like parts throughout the drawings, wherein:

FIG. 1 is a schematic diagram of an exemplary dual mode endoscope in accordance with aspects of the present technique;

FIG. 2 is a schematic diagram of detecting scattered and/or emitted light from two or more optical imaging modalities in accordance with one aspect of the present technique;

FIG. 3 is a schematic diagram of detecting scattered and/or emitted light from two or more optical imaging modalities in accordance with another aspect of the present technique; and

FIG. 4 illustrates images from two different optical imaging modalities and a combined image of the types obtainable by the present techniques.

### DETAILED DESCRIPTION

The present techniques and devices are generally directed to multi-mode optical imaging systems using a single detector. Generally, the technique may be employed in a variety of medical and non-medical imaging contexts. Though the present discussion provides examples in context of flexible or rigid endoscopy, one of ordinary skill in the art will readily comprehend that the application of the techniques in other contexts, such as for borescopy and/or microscopy, is within the scope of the present techniques.

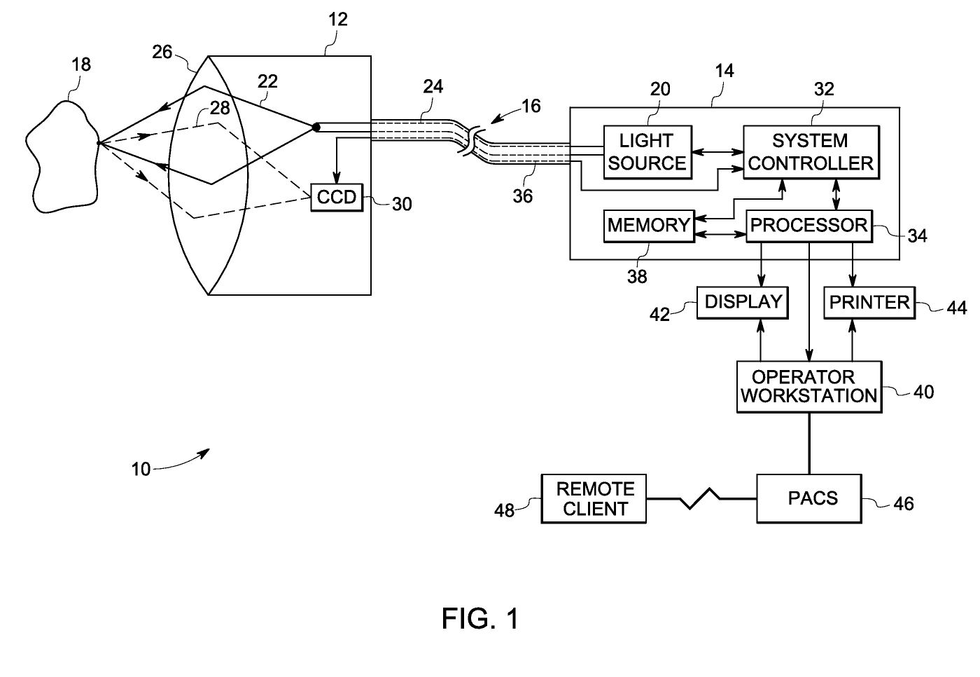

Referring now to FIG. 1, a schematic diagram of an exemplary multi-mode endoscope 10 is illustrated in accordance with aspects of the present technique. In the illustrated embodiment, the endoscope 10 includes a probe 12 and a body 14. The probe 12 may be coupled to the body 14 via a flexible cable 16. The probe 12 is guided inside a cavity to be inspected, such as digestive or respiratory tract of a human body towards a specimen 18 to be imaged or examined during a medical procedure. One skilled in the art will appreciate that anatomical subjects as well as luggage, packages, articles of manufacture, and the like may be inspected using the exemplary multi-mode endoscope 10.

The body 14 includes one or more illumination sources 20 for emitting light corresponding to two or more optical imaging modalities and directing the emitted light 22 towards the specimen 18 (i.e., the section of the body to be examined) via a light delivery and collection subsystem. It should be noted that, in certain embodiments, the light corresponding to two or more optical imaging modalities may be multiplexed in time. The multiplexing or interleaving of the light may be performed automatically and in real-time with minimal or no manual intervention. Such automated and/or real-time multiplexing of light corresponding to two or more optical imaging modalities may not be useful even if employed by conventional techniques without employing a detector and/or detection schemes as will be described in the various embodiments below. The light delivery and collection subsystem may include fiber optic cables 24 and one or more optical devices 26 (e.g., lenses, prisms, mirrors, and so forth). The illumination source 20 may be any broadband source such as light-emitting diodes, super-luminescent diodes, broadened laser sources, tunable light sources, monochromatic light source, polychromatic light source, and so forth. Any optical imaging modalities may be employed including a white light imaging, a narrowband brightfield imaging, a luminescence imaging, or a near infrared imaging.

In certain embodiments, the illumination sources 20 illuminate the specimen with a visible light and an excitation light. The excitation light may be a wavelength selected to induce luminescence in the specimen via intrinsic luminescence. Alternatively, the excitation light may be a wavelength selected to induce luminescence in a luminescence agent administered to the subject so as to come into contact with the specimen. As noted above, in certain embodiments, the visible light and the excitation light may be multiplexed in time.

The specimen 18 may scatter or emit light 28 detectable by two or more optical modalities upon being illuminated by the light 22. As noted above, the light may be emitted from the specimen 18 via agent-induced luminescence or auto-luminescence. The light emitted by luminescence may be in near infrared spectral region or in near ultraviolet spectral region based on the specimen and the type of luminescence agent administered into the specimen. The scattered and/or emitted light 28 may be detected via a single detector 30, such as a CCD detector or a CMOS detector. Any known collection mechanism may be employed by present technique to collect the scattered and/or emitted light 28 from the specimen 18 and deliver the same to the detector 30. In certain embodiments, the detector 30 may be disposed within the probe 12 (distal end of the endoscope). Alternatively, the detector 30 may be disposed within the body 14 (midsection or proximal end of the endoscope) and configured to receive the emitted or scattered light 28 from the specimen 18 through the light delivery and collection subsystem. In addition to the fiber optic cables 24 and the optical devices 26, the light delivery and collection subsystem may also include a notch or a cut filter (not shown) disposed adjacent to the detector 30 on a light-incident side and configured to block the scattered (reflected) excitation light.

A single detector 30 may be adapted to detect scattered and/or emitted light 28 coming from the specimen 18 and detectable by each of the two or more optical imaging modalities in accordance with aspects of the present technique. For example, the single detector detects white light reflected from the specimen and luminescent light emitted via luminescence and generates a detector output signal in response to the detected light. The detector 30 is generally formed by a plurality of detector elements (cells), which detect the scattered, reflected and/or emitted light detectable by each of the two or more optical imaging modalities. For example, the detector 30 may include multiple rows and/or columns of detector elements arranged in a two-dimensional array. Each detector element, when impacted by a light flux, produces an electrical signal proportional to the absorbed light flux at the position of the individual detector element in detector 30. These signals are acquired through read-out electronics or data readout circuitry (not shown) coupled to the detector cells. The signals may then be processed to reconstruct or generate an image of the specimen 18, as described below.

The illumination sources 20 is controlled by a system controller 32, which furnishes power, control signals and so forth for examination sequences. For example, in certain embodiments, the system controller 32 may multiplex the visible light and an excitation light in time via a multiplexer (not shown). As will be appreciated by those skilled in the art, multiplexing is transferring multiple signals (e.g., light detectable by different modalities) apparently simultaneously as sub-channels in one communication channel. In one embodiment, signals may be multiplexed using time-division multiplexing, in which the multiple signals are carried over the same channel in alternating time slots.

Moreover, the detector 30 is coupled to the system controller 32, which controls the acquisition of the signals generated in the detector 30. The system controller 32 may also execute various signal processing and filtration functions, such as for initial adjustment of dynamic ranges, interleaving of digital image data, and so forth. In general, system controller 32 commands operation of the endoscope 10 to execute examination protocols and to process acquired data. In the present context, system controller 32 may also include signal-processing circuitry, that may be based upon a general purpose or application-specific digital computer, and associated memory circuitry. The associated memory circuitry may store programs and routines executed by the computer, configuration parameters, and image data. For example, the associated memory circuitry may store programs or routines for reconstructing image from the detector output signal.

The system controller 32 may include data acquisition circuitry (not shown) for receiving data collected by readout electronics of the detector 30. In particular, the data acquisition circuitry typically receives sampled analog signals from the detector 30 and converts the data to digital signals for subsequent processing by a processor 34. The detector output signal may be transmitted to the system controller 32 over a wired or a wireless link 36.

The processor 34 is typically coupled to the system controller 32 and may include a microprocessor, digital signal processor, microcontroller, as well as other devices designed to carry out logic and processing operations. The data collected by the data acquisition circuitry may be transmitted to the processor 34 for subsequent processing such as reconstruction. For example, the data collected from the detector 30 may undergo pre-processing and calibration at the data acquisition circuitry within system controller 32 and/or the processor 34 to condition the data to represent the specimen 18. The processed data may then be reordered, filtered, and reconstructed to formulate an image of the imaged area. Once reconstructed, the image generated by the endoscope 10 reveals the specimen 18 which may be used for diagnosis, evaluation, and so forth.

The processor 34 may comprise or communicate with a memory 38 that can store data processed by the processor 34 or data to be processed by the computer 34. It should be understood that any type of computer accessible memory device capable of storing the desired amount of data and/or code may be utilized by such an exemplary multi-mode endoscope 10. Moreover, the memory 38 may comprise one or more memory devices, such as magnetic or optical devices, of similar or different types, which may be local and/or remote to the endoscope 10. The memory 38 may store data, processing parameters, and/or computer programs comprising one or more routines for performing the reconstruction processes. Furthermore, memory 38 may be coupled directly to system controller 32 to facilitate the storage of acquired data.

The processor 34 may also be adapted to control features enabled by the system controller 32, for example, acquisition. Furthermore, the processor 34 may be configured to receive commands from an operator via an operator workstation 40 which may be equipped with a keyboard or other input devices. An operator may thereby control the endoscope 10 via the operator workstation 40. The operator may observe the reconstructed image and other data relevant to the system from operator workstation 40, initiate imaging, and otherwise control the system.

The endoscope 10 may be equipped with or connectable to a display unit 42 or a printer 44. The display unit 42 coupled to the operator workstation 40 may be utilized to observe the reconstructed image. In one embodiment, the image may be displayed at a near video rate. Additionally, the image may be printed by the printer 44 coupled to the operator workstation 40. The display 42 and the printer 44 may also be connected to the processor 34, either directly or via the operator workstation 40. Further, the operator workstation 40 may also be coupled to a picture archiving and communications system (PACS) 46. It should be noted that PACS 46 might be coupled to a remote system 48, such as a radiology department information system (RIS), hospital information system (HIS) or to an internal or external network, so that others at different locations may gain access to the image data.

One or more operator workstations 40 may be linked in the system for system controlling functions such as outputting system parameters, requesting examinations, viewing images. In general, displays, printers, workstations, and similar devices supplied with the system may be local to the data acquisition components, or may be remote from these components, such as elsewhere within an institution or hospital, or in an entirely different location, linked to the endoscope via one or more configurable networks, such as the Internet or virtual private networks.

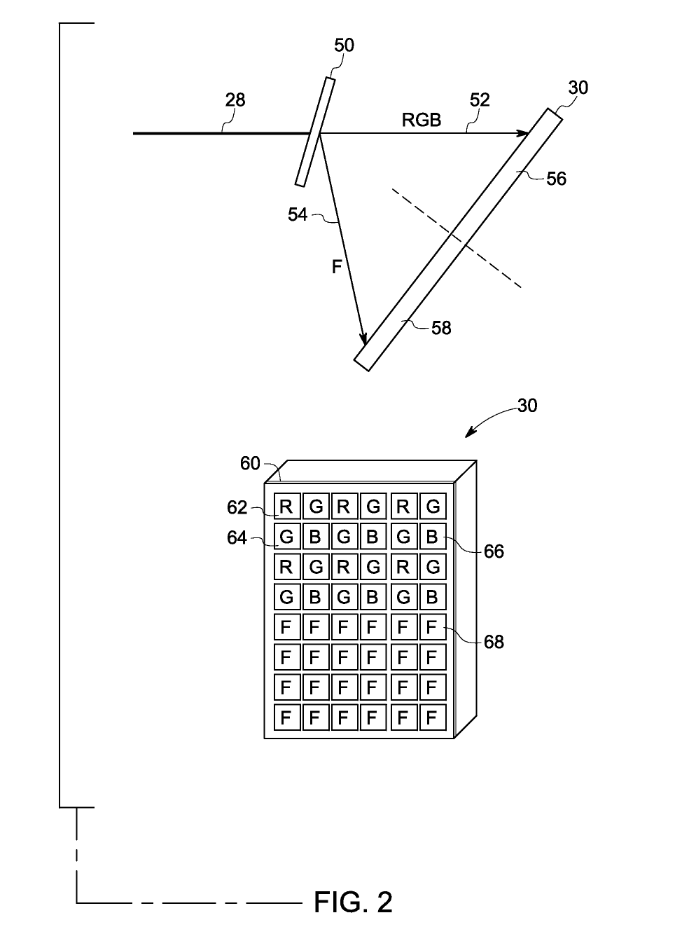

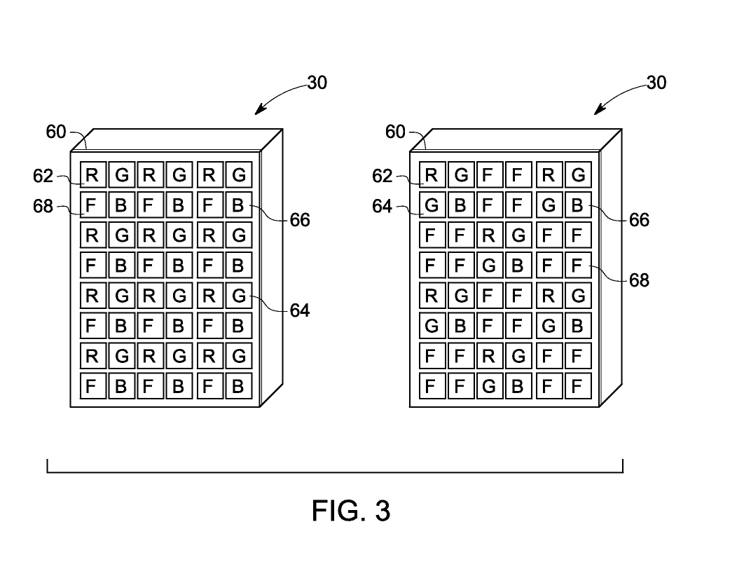

The exemplary endoscope 10, as well as other multi-mode optical imaging systems may employ multi-mode detectors, such as the single detector 30, adapted to detect light from two or more optical imaging modalities. The single detector 30 may employ a variety of detection schemes to detect light from two or more optical imaging modalities. For example, the single detector 30 may detect the light detectable by each of the two or more optical imaging modalities simultaneously or sequentially. FIGS. 2 and 3 illustrate some of the variety of configurations that may be employed to enable the single detector 30 to detect light detectable by two or more optical imaging modalities simultaneously. As illustrated, the detector 30 may be split or divided into distinct regions each dedicated to respective optical imaging modalities. Alternatively, custom filters (onboard or separately attached) may be employed to transmit light detectable by one of the modalities at any given pixel of the detector 30.

As depicted into FIG. 2, a plurality of filters 60 may be placed adjacent to at least one of the regions of the single detector 30 so as to split the light 28 and transmit the light of a particular optical imaging modality (e.g., luminescent or white light) to the corresponding region of the detector. The plurality of filters may include filters corresponding to the three basic color components of white light such as a red filter (R) 62, a green filter (G) 64, and a blue filter (B) 66. Alternatively, the plurality of filters may include filters corresponding to the three complementary color components of white light such as a cyan filter (C), a magenta filter (M), and a yellow filter (Y). The detector may further include a filter for the non-white light applications such as a luminescent filter (F) 68.

Additionally, in certain embodiments, one or more optical devices 50, such as a dichroic mirror or a beam splitter, may be employed by for splitting the scattered and/or emitted light 28 and directing light 52, 54 from respective optical imaging modalities to the corresponding regions 56, 58 in the single detector 30. Thus, one of the regions of the detector 58 may receive emitted light 54 from the luminescent light source while the other region 56 may receive the scattered light 52 from the white field light source. In the illustrated embodiment, the luminescence-transmissive elements (luminescent filters) may be positioned on half of the array (upper/low or left/right). Thus, half of the detector 30 is dedicated to luminescence using either onboard or off board filters 60 and/or by spectral separation of the two channels prior to detection. It should be noted that, in these spilt embodiments, a dichroic element positioned between the item of interest and the sensor array may be used to direct the white light to the non-luminescent-transmissive elements (non-luminescent filters) and the luminescent light to the luminescence-transmissive elements (luminescent filters). Further, it should be noted that the illumination may be done simultaneously or sequentially.

Alternatively, as illustrated in FIG. 3, the single detector may include a plurality of detector cells and onboard filters or off-board filters positioned adjacent to the plurality of detector cells such that different detector cells detect light from different optical imaging modalities. In certain embodiments, the filters may be arranged in a repeating N×M pattern where at least one of the dimensions N or M is greater than or equal to two. The filters may include monochromatic filters, color filters, or luminescent filters. For example, the filters 60 may include a custom Bayer-type filter where one of the two green pixels has been replaced with a luminescence emission filter. Alternatively, the onboard or off-board filters may be customized such that the two or more channels may be arranged to match a variety of filter patterns. For example, a checkerboard pattern may be used to facilitate registration of the two or more channels where half the pixels are dedicated to each channel. As noted above, the custom onboard or off-board (external) filters may be such that one of the two green filters in Bayer-type color filter may be replaced by a luminescent filter. Thus, the single detector is adapted to capture light from two or more imaging modalities simultaneously so that different pixels in the single detector may be adapted to receive light from different modalities.

Although the filter arrays are depicted in the figures as 8×6 matrices of repeating 2×2 or repeating 2×4 patterns, the general patterns may be applied to other configurations. In one embodiment, a 2×2 or a 2×4 pattern of selectively transmissive elements may include repetition of green, red, blue, and luminescence-transmissive elements (green, red, blue and luminescent filters). Alternatively, a 2×2 or a 2×4 pattern of selectively transmissive elements may include repetition of cyan, magenta, yellow, and luminescence transmissive elements (cyan, magenta, yellow, and luminescent filters). Alternatively, in certain embodiments, a 2×1 pattern of selectively transmissive elements may include repetition of monochromatic (gray) and luminescence-transmissive elements (monochromatic and luminescent filters).

In each of the embodiments described above, total number of luminescence transmissive elements (luminescent filters) and, consequently, the number of dedicated pixels, may be increased or decreased based on the spectral characteristics of the object of interest such as emission spectra, intensity, abundance of signal, and desired spatial resolution of the specimen.

In certain embodiments, a polychromatic or color filter may be coupled to a monochromatic detector for enabling the detector and hence a device employing such detectors to detect or extract polychromatic information from two or more optical imaging modalities. The polychromatic filter may include a plurality of monochromatic filters (red, green, blue, cyan, magenta, yellow or luminescent) arranged in a pattern so as to transmit light detectable by two or more optical imaging modalities. Thus, the plurality of monochromatic filters may be split into distinct regions each dedicated to transmit light from the respective optical imaging modalities. The plurality of monochromatic filters form a pattern such that each pattern is capable of transmitting light from each of the two or more optical imaging modalities. Each of the patterns may include a red filter, a blue filter, a green filter, and a luminescence filter. Alternatively, each of the patterns may include a cyan filter, a magenta filter, a yellow filter, and a luminescence filter. The polychromatic filters may be coupled to the monochromatic detectors or may be integrated to the detectors to form a polychromatic detector (color detector).

Thus, the device for extracting polychromatic information from a monochromatic detector may include a polychromatic filter assigned to each of a plurality of detector cells of the monochromatic detector. The polychromatic filter is configured to transmit light corresponding to two or more optical imaging modalities to the monochromatic detector. As noted above, polychromatic information comprises information from at least two of a white light imaging modality, a narrowband brightfield imaging modality, a luminescence imaging modality, or a near infrared imaging modality. The device sequentially illuminates a sample with light from two or more optical imaging modalities. Further, the device sequentially reads reflectance and/or emittance with a monochromatic detector through the polychromatic filter. Such a device may further include a processor for digitally combining the polychromatic information into a multicolored image.

The process of extracting polychromatic information from the single monochromatic detector involves assigning to individual pixels in the detector a fixed RGBF/CMYF filter (i.e., a polychromatic filter), illuminating the specimen with white light and excitation light simultaneously or sequentially in alternating frames or sets of frames, and detecting the scattered or emitted light from the specimen with a monochromatic detector with RGBF/CMYF filters applied to it.

Similarly, a variety of techniques may be employed to enable the single detector 30 to detect light corresponding to two or more optical imaging modalities sequentially. For example, in certain embodiments, the illumination sources alternate frames between two modalities (i.e., the light from two or more modalities may be multiplexed in time) and each of a plurality of detector cells of the single detector is sensitive to each of the two or more optical imaging modalities. To enable this, the source of illumination and the detector are typically synchronized with respect to each other. For example, the one or more illumination source and the single detector may be phase locked (i.e., locked in phase) or synchronized with respect to each other. The gain of the readout circuitry of the single detector is then synchronously altered based on detection requirements of light from respective optical imaging modalities. For example, the gain of the readout electronics may be set to normal when sensing the white light and may be increased when sensing luminescent light along with the synchronization of the luminescent light source. Thus, as will be appreciated by those skilled in the art, the same detector and filters as currently used may be employed with changes in the readout electronics to impart dual mode detection functionality. The change in the readout circuitry may be performed by a separate processing chip in the CCD detector or may be integrated in the CMOS detector. Further, it should be noted that the multiplexing and the gain change may be performed in a single automated acquisition. Changes in gain may be performed on individual pixels or regions, when such pixels or regions are specifically modality sensitive (e.g., to wavelengths of luminescence).

The process involves illuminating the specimen with light from two or more time-multiplexed modalities (alternating between modalities). For example, the specimen may be illuminated by alternating between white light and luminescent excitation light. The process further involves capturing the scattered and/or emitted light through the single detector by synchronization and gain changes. It should be noted that the technique may still employ color detectors or custom onboard color filters along with the monochromatic detector for sensing the light corresponding to two or more optical imaging modalities.

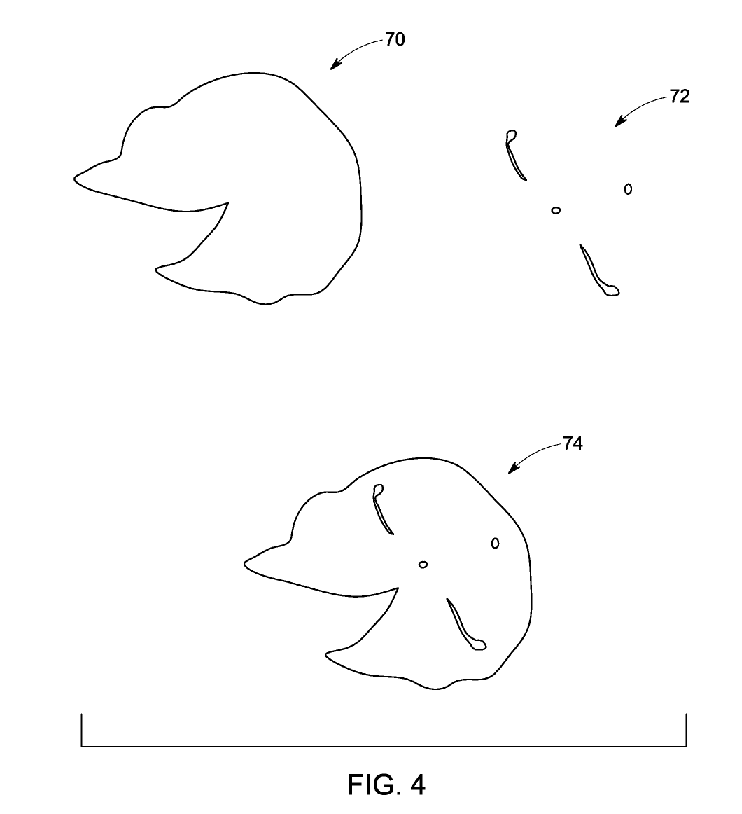

FIG. 4 illustrates images that may be obtained by employing the multi-mode endoscope described in the various embodiments discussed above. For example, a white light image 70 may be combined with a luminescent image 72 to get a combined image 74 via the multi-mode endoscope described in the embodiments discussed above.

The techniques described in various embodiments discussed above provide multi mode functionality in an multi-mode optical imaging systems via a single detector chip (i.e., no separate dedicated detectors are required for each optical imaging modalities). The technique enables simultaneous capturing of images using different portions of the detector in combination with mask filters or sequential capturing of images using a single detector with alternating illumination and detection patterns. It should be noted that no manual switching is required for altering the gain of the readout circuitry during sequential detection. In one embodiment, the technique enables capturing both reflected light (e.g., visible or near infrared light) and luminescence (e.g., visible or near infrared light) using of a single detector.

The consolidation of multiple detection channels onto a single detector reduces the size of the image acquisition devices, which is particularly beneficial for minimally invasive surgical devices such as dual mode endoscopes. In particular, the consolidation greatly reduces the size of the probe (in distal end approach) and thus increases patient comfort, thereby causing little or reduced discomfort to patient. In other words, the use of single detector enables miniaturization of the endoscope. Thus, applications requiring lower diameter endoscopes, such as upper GI and lung, would benefit from the techniques described above. Additionally, the use of single detector for receiving light from different modalities eliminates problems associated with image registration that occur when multiple detectors are used to capture optical images. Moreover, in certain embodiments, the techniques enable collection of light from two or mode optical imaging modalities (white light and luminescent light) simultaneously and in real time. Further, the dual mode endoscope discussed in the embodiments discussed above may be coupled with a dedicated video processor and contrast agents specific to different clinical applications for enhanced imaging and diagnosis.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A system for multi-mode optical imaging, the system comprising:
one or more illumination sources for directing a visible light and an excitation light multiplexed in time towards a specimen, the excitation light configured to induce luminescence in the specimen; and
a single detector in synchronization with the one or more illumination sources for detecting visible light scattered or reflected from the specimen and luminescent light emitted via luminescence.

2. The system of claim 1, wherein the single detector comprises a plurality of detector cells and wherein at least some of the plurality of detector cells are sensitive to the visible light and the luminescent light.

3. The system of claim 1, further comprising a readout circuitry for the single detector, the readout circuitry configured to acquire signals from the single detector for each of the detected visible light and the detected luminescent light sequentially and to generate a detector output signal in response to the detected light.

4. The system of claim 3, wherein the readout circuitry has a gain that is automatically and synchronously altered based on detection requirements of the visible light and the luminescent light.

5. The system of claim 4, wherein multiplexing of the visible light and the excitation light and alteration of gain of the readout circuitry is performed in a single automated acquisition.

6. The system of claim 1, wherein the single detector is a CCD based detector or a CMOS based detector.

7. The system of claim 1, further comprising a processor for reconstructing an image from a detector output signal generated by the single detector.

8. The system of claim 7, further comprising a display for displaying the reconstructed image at a near video rate during acquisition of signal from the single detector.

9. The system of claim 1, further comprising an optical fiber configured to deliver light from the one or more illumination sources to the specimen.

10. The system of claim 1, further comprising a system controller for controlling the operation of the one or more illumination sources, the single detector, or a combination thereof.

11. A multi-mode endoscope, comprising:
one or more illumination sources disposed within an endoscope body and configured to direct a visible light and an excitation light multiplexed in time towards a specimen via a fiber optic cable, the excitation light configured to induce luminescence in the specimen; and
a single detector disposed within an endoscope probe, in synchronization with the one or more illumination sources, and configured to detect visible light scattered or reflected from the specimen and luminescent light emitted via luminescence.

12. The multi-mode endoscope of claim 11, wherein the single detector comprises a plurality of detector cells and wherein at least some of the plurality of detector cells are sensitive to the visible light and the luminescent light.

13. The multi-mode endoscope of claim 11, further comprising a readout circuitry for the single detector, the readout circuitry configured to acquire signals from the single detector for each of the detected visible light and the detected luminescent light sequentially and to generate a detector output signal in response to the detected light.

14. The multi-mode endoscope of claim 13, wherein the readout circuitry has a gain that is automatically and synchronously altered based on detection requirements of the visible light and the luminescent light.

15. The multi-mode endoscope of claim 14, wherein multiplexing of the visible light and the excitation light and alteration of gain of the readout circuitry is performed in a single automated acquisition.

16. A method for multi-mode optical imaging, the method comprising:
directing a visible light and an excitation light multiplexed in time towards a specimen, the excitation light configured to induce luminescence in the specimen; and
detecting visible light scattered or reflected from the specimen and luminescent light emitted via luminescence via a single detector that is in synchronization with the one or more illumination sources.

17. The method of claim 16, further comprising acquiring signals from the single detector for each of the detected visible light and the detected luminescent light sequentially and generating a detector output signal in response to the detected light via a readout circuitry for the single detector.

18. The method of claim 17, wherein acquiring signals comprises automatically and synchronously altering a gain of the readout circuitry based on detection requirements of the visible light and the luminescent light.

19. The method of claim 18, wherein multiplexing of the visible light and the excitation light and alteration of gain of the readout circuitry is performed in a single automated acquisition.

20. The method of claim 16, further comprising reconstructing an image from a detector output signal generated by the single detector.
